(19) United States
(12) Patent Application Publication
(10) Pub. No.: US 2024/0336775 A1
(43) Pub. Date: Oct. 10, 2024

(54) METHOD FOR PREPARING AN ENZYME MASTERBATCH

(71) Applicant: CARBIOLICE, RIOM (FR)

(72) Inventors: Chloe GUILLAUMONT, Chambaron sur Morge (FR); Clementine ARNAULT, Clermont Ferrand (FR); Yannick CHARPENTIER, Aigueperse (FR)

(21) Appl. No.: 18/579,979

(22) PCT Filed: Jul. 20, 2022
(86) PCT No.: PCT/EP2022/070302

§ 371 Date: Jan. 17, 2024

(30) Foreign Application Priority Data
Jul. 20, 2021 (FR) FR2107809

(51) Int. Cl. C08L 67/04; B32B 27/08; B32B 27/36; C08J 3/22; C08K 3/26

(52) U.S. Cl. CPC C08L 67/04; B32B 27/08; B32B 27/36; C08J 3/226; C08K 3/26; B32B 2250/24; B32B 2250/40; B32B 2307/7163; C08J 2367/04; C08J 2405/00; C08K 2003/265; C08L 2201/06; C08L 2310/00

(57) ABSTRACT

The invention relates to a method for preparing a masterbatch (or “masterbatch”) comprising a polysaccharide, enzymes and a low-melting polymer in a mixer. In particular, this masterbatch is used for the manufacture of items made of a biodegradable plastic material.

### FIELD OF THE INVENTION

The invention relates to a method for preparing a masterbatch (or “masterbatch”) comprising a polysaccharide, enzymes and a low-melting polymer in a mixer. In particular, this masterbatch is used for the manufacture of items made of a biodegradable plastic material.

### PRIOR ART

Methods for preparing plastic materials based on biodegradable and bio-sourced polyesters have been developed in order to address ecological challenges. These plastic products, synthesised from starch or starch derivatives and polyester, are used for the manufacture of items having a short lifespan, such as plastic bags, food packaging, bottles, wrapping films, etc.

In general, these plastic compositions contain polyester and flours originating from various cereals (U.S. Pat. Nos. 5,739,244; 6,176,915; US 2004/0167247; WO 2004/113433; FR 2 903 042; FR 2 856 405).

In order to control the degradation of these plastic products, the addition of one or more additive(s) like mineral fillers (WO 2010/041063) and/or biological entities having a polyester degrading activity (WO 2013/093355; WO 2016/198652; WO 2016/198650; WO 2016/146540; WO 2016/062695) has been suggested.

Thus, biodegradable plastic items comprising biological entities, more particularly enzymes dispersed in a polymer, have a better biodegradability compared to plastic products free of these enzymes.

Methods for preparing these enzymatic plastics have been previously described, however problems related to homogeneity and roughness might appear and affect the physical properties of the product. For example, the presence of enzyme aggregates results in a greater roughness, the aesthetics of the product are reduced and the physical and mechanical properties are altered. A first improvement has been made by adding the enzyme, mixed beforehand with a polysaccharide and a solvent in one single liquid formulation, to the support polymer (WO 2019/043145, WO 2019/043134).

An object of the invention is to facilitate and secure the industrial implementation of the masterbatch preparation method, in particular by simplifying the tools required for implementation thereof while preserving, and possibly improving, their performances in terms of preservation of the activity of the enzymes after formulation, and of ability to be used in the preparation of final items.

The present invention describes a method for preparing a masterbatch, which when used in the manufacture of plastic products comprising enzymes dispersed in a polymer, allows improving the dispersion of the enzymes in the final compound as well as the level of biodegradability of the plastic material without modifying the mechanical properties of the product.

### DISCLOSURE OF THE INVENTION

The present invention relates to a method for preparing a masterbatch comprising a polysaccharide, enzymes and a support polymer in a mixer, said method comprising the following steps:
a) separately and simultaneously introducing a1) the enzymes in the solution, a2) the polysaccharide and a3) the support polymer,
b) mixing them at a temperature at which the support polymer is partially or totally molten, and
c) recovering the masterbatch.

The invention also relates to masterbatches thus obtained and plastic items obtained by mixing the masterbatch with a polymer or a mixture of polymers comprising a polymer capable of being degraded by the enzymes of the masterbatch.

In particular, it relates to a method for preparing a plastic item comprising a polymer that could be degraded by enzymes and enzymes capable of degrading said polymer, comprising a step of mixing the masterbatch according to the invention with said polymer, alone or in a mixture.

### DETAILED DESCRIPTION OF THE INVENTION

The present invention relates to a method for preparing a masterbatch comprising a polysaccharide, enzymes capable of degrading polyesters and a support polymer in a mixer, said method comprising the following steps:
a) separately and simultaneously introducing the liquid formulation of enzymes, the polysaccharide and the support polymer,
b) and mixing them at a temperature at which the support polymer is partially or totally molten, and
c) recovering the masterbatch.

Unless indicated otherwise, the percentages are given by weight with respect to the total weight of the composition to which they refer.

As is used here, the term “polysaccharides” refers to molecules composed of long chains of monosaccharide units bonded together by glycosidic bonds. The structure of the polysaccharides may be linear to strongly branched. Examples include storage polysaccharides such as starch and glycogen, and structural polysaccharides such as cellulose and chitin. Polysaccharides comprise native polysaccharides or polysaccharides that are chemically modified by cross-linking, oxidation, acetylation, partial hydrolysis, etc.

Carbohydrate polymers may be classified according to their source (marine, plant, microbial or animal), the structure (linear, branched) and/or a physical behaviour (such as the designation as gum or hydrocolloid which refers to the property that these polysaccharides hydrate in hot or cold water to form viscous solutions or dispersions at a low gum or hydrocolloid concentration).

In the context of the invention, polysaccharides may be classified according to the classification described in “Technologies d'encapsulation pour ingredients actifs alimentaires et transformation alimentaire—Chapter 3—Materiaux pour encapsulation—by Christine Wandrey, Artur Bartkowiak and Stephen E. Harding”:
Starch and derivatives, such as amylose, amylopectin, maltodextrin, glucose syrups, dextrin, and cyclodextrin
Cellulose and derivatives, such as methylcellulose, hydroxypropyl methylcellulose, ethylcellulose, etc.
Plant exudates and extracts, also called plant gums or natural gums, including, yet without limitation, gum arabic (or acacia gum), tragacanth gum, guar gum, locust bean gum, karaya gum, mesquite gum, galactomannans, pectin, soluble soybean polysaccharide
Marine extracts such as carrageenan and alginate
Microbial and animal polysaccharides such as gellan, dextran, xanthan and chitosan.

Polysaccharides may be classified according to their solubility in water. In particular, cellulose is not soluble in water. According to the invention, polysaccharides have the ability to be soluble in water.

The polysaccharides used in the formulation of plastic compositions are well-known to a person skilled in the art. In particular, they are selected from among starch derivatives like amylose, amylopectin, maltodextrins, glucose syrup, dextrins and cyclodextrins, natural gums like gum arabic, tragacanth gum, guar gum, locust beam gum, gum karaya, mesquite gum, galactomannans, pectin or soluble soybean polysaccharides, marine extracts like carrageenans and alginates, and microbial or animal polysaccharides like gellans, dextrans, xanthans or chitosan, and mixtures thereof.

The polysaccharide may also be a mixture of several ones of the aforementioned polysaccharides.

In a preferred embodiment, the used polysaccharide is a natural gum, and more particularly gum arabic.

The used enzymes are enzymes having a polyester degrading activity. Thus, their incorporation in products made of biodegradable polyester-based plastic improves the biodegradability of these.

Examples of enzymes having a polyester degrading activity are well-known to a person skilled in the art, in particular depolymerases, esterases, lipases, cutinases, carboxylesterases, proteases or polyesterases.

In particular, mention will be made of enzymes capable of degrading polyesters so as to improve the biodegradability of the items prepared with the masterbatch according to the invention. In a particular embodiment of the invention, the enzymes are capable of degrading PLA. Such enzymes and the method of incorporation thereof in thermoplastic items are known to a person skilled in the art, in particular described in the patent applications WO 2013/093355, WO 2016/198652, WO 2016/198650, WO 2016/146540 and WO 2016/062695.

In particular, the enzymes used in the context of the invention are selected from among proteases and serine proteases. Examples of serine proteases include Proteinase K of Tritirachium album, or PLA-degrading enzymes derived from Amycolatopsis sp., Actinomadura keratinilytica, Laceyella sacchari LP175, Thermus sp., or Bacillus licheniformis or reformulated commercial enzymes known to be PLA-degrading such as Savinase®, Esperase®, Everlase® or any enzyme from the family of subtilisins CAS [9014-01-1] or any functional variant.

The enzymes may be used in their pure or enriched form, and possibly mixed with one or more excipient(s).

In particular, the enzymes are selected so as to be capable of degrading at least one polymer of a plastic item that will be obtained using the masterbatch in its manufacturing process.

The enzymes are used in the method according to the invention in the form of an liquid solution. A liquid formulation according to the invention is an enzymatic solution and/or an suspension of enzymes in a solvent, in particular a thick suspension, capable of flowing at room temperature. The liquid formulation should be in a form suited to be introduced in the mixer by any usual means for introducing a liquid formula in a mixer. Thus, the formulation may be introduced via an injector or a peristaltic pump. A person skilled in the art should be able to determine which device is the most suitable for the addition of the formulation. In a preferred embodiment, the liquid formulation is introduced via a peristaltic pump. The solvent is a solvent that does not degrade the enzymes, and more particularly water. According to an embodiment of the invention, the liquid formulation essentially consists of enzymes and of the solvent, in particular water.

In particular, the form of the formulation will depend on the enzymes content. It should be understood that the enzymes content exceeds the solubility threshold of these in the solvent, the formulation will comprise enzymes in suspension and will look like a thick composition, yet capable of flowing. According to an advantageous embodiment of the invention, the liquid enzymatic formulation is an enzymatic solution.

According to one embodiment, the liquid enzymatic formulation comprises from 0.01 to 70% by weight of enzymes, in particular from 0.3 to 60% of enzymes, more particularly from 0.5 to 35% of enzymes. In particular, the enzymatic formulation, in particular the enzymatic solution, may comprise an enzymes content of 1%, 5%, 10%, 15%, 20%, 25%, 30%, 35% or more by weight.

The support polymer is a low-melting polymer and a polymer that advantageously has a melting point lower than 140° C. and/or a glass transition temperature lower than 70° C. It should also be compatible with the one or more polymer(s) with which the masterbatch will be mixed for the preparation of the enzymatic plastic items.

Such support polymers are well-known to a person skilled in the art. In particular, these consist of polycaprolactone (PCL), polybutylene succinate (PBS), polybutylene succinate adipate (PBSA), polybutylene adipate terephthalate (PBAT), polydioxanone (PDS), polyhdroxyalkanoate (PHA), polylactic acid (PLA), or copolymers thereof. These may also consist of natural polymer like starch or a polymer that is described as universal, in other words compatible with a wide range of polymers like an EVA-type copolymer.

Advantageously, the support polymer has a melting point lower than 120° C. and/or a glass transition temperature lower than 30° C.

In general, the support polymer is one single polymer as defined hereinabove. It may also consist of a mixture of these support polymers.

According to a particular embodiment of the invention, the support polymer is PCL.

According to a particular embodiment of the invention, the enzymes of the masterbatch are not capable of degrading the support polymer.

Step a) corresponds to the addition of the polysaccharide, the enzymes in liquid formulation and the support polymer in the mixer. The polysaccharide is in a pulverulent form and is introduced in the mixer using a batcher specific for powders. The enzymes in liquid formulation are added by any common means for introducing a solution in a mixer or any other industrial means like a peristaltic pump or an injector. In one embodiment, the enzymes in liquid formulation are added via a peristaltic pump. The support polymer is in the form of granules and is introduced in the mixer via a batcher specific for granules.

According to a particular embodiment, the masterbatch is prepared by mixing:
60 to 90% of support polymer, in particular PCL,
10 to 20% of liquid enzymatic formulation, in particular enzymatic solution,
2 to 15% of polysaccharide, in particular gum arabic.

According to a particular embodiment of the invention, the masterbatch comprises at most 5% of enzymes having a polyester degrading activity. Hence, the liquid enzymatic formulation, in particular the enzymatic solution, content used in the manufacture of the masterbatch will depend on the enzyme content in its liquid formulation.

Advantageously, the liquid enzymatic formulation/polysaccharide ratio is calculated so as to have a dry mass (enzymes and polysaccharide) from 30% to 70% of the mixture of both.

The polysaccharide/enzymatic solution ratio is determined so as to have a dry mass of at least 30% and at most 55% and possibly at most 70%.

A person skilled in the art will know how to adapt the characteristics of the process (temperature and time) necessary for carrying out step a) according to the used components (polysaccharide, enzymes and support polymer).

Mixing of step b) is done at a temperature at which the support polymer is partially or totally molten. It should be understood that the temperature of step b) will be determined by a person skilled in the art so that it does not alter the enzymes, and more particularly does not substantially reduce their support polymer degrading enzymatic activity. According to a particular embodiment, the temperature of step b) is a temperature lower than or equal to the melting point (Tm) of the support polymer. A person skilled in the art could select the support polymer according to its melting point and the capacity of the selected enzymes to withstand this temperature in the method according to the invention. In a particular embodiment, the temperature of step b) is comprised between the glass transition temperature (Tg) and the melting point (Tm) of the support polymer. Alternatively, the temperature of step b) is set at a temperature equal to or above the Tm of the support polymer.

In general, the temperature of step b) may be comprised between 40 and 200° C. In one embodiment, the temperature is higher than 40° C., or higher than 50° C. Preferably, the temperature is comprised between 55 and 175° C. In a preferred embodiment, the temperature of step b) is adjusted according to the nature of the used polymer. Thus, the temperature of step b) is at or generally corresponds to the melting point of the used polymer. Typically, the temperature does not exceed 300° C., more particularly, the temperature does not exceed 250° C.

It is sought to maintain a temperature of the mixture in step b) which is the lowest possible enabling mixing and homogeneous dispersion of the enzymes and of the polysaccharide in the support polymer.

In a particular embodiment, the support polymer is PCL and the mixing temperature of step b) is about 60° C., from 55 to 65° C.

Mixing of the polysaccharide, enzyme and support polymer components in step b), is performed for a time period of 10 to 35 seconds. In a particular embodiment, mixing lasts between 15 and 35 seconds, in particular for about 20 seconds, for about 25 seconds or for about 30 seconds.

Throughout the process for producing the masterbatch, the temperature is gradually increased in order to ensure a homogeneous and constant mixing while preserving the characteristics and properties of each of the components as best as possible.

Advantageously, the stay time of the polysaccharide/enzymes composition in the polymer at a temperature above 100° C. within the mixer (steps b) and c)) is as short as possible. Preferably, it is comprised between 5 seconds and 10 minutes. However, a stay time of less than 5 minutes is preferred. In a preferred embodiment, the latter is less than 3 minutes, and possibly less than 2 minutes.

The masterbatch obtained in step c) is in a solid form. Advantageously, it is recovered in the form of granules. These granules could be stored, transported, and incorporated in the manufacture of plastic products or items, regardless of their shape and their use, which could be called “end products”. These may consist of films, or flexible or solid parts with shapes and volumes suited for their uses.

The formulation of the masterbatch may comprise a mineral filler. In this case, the mineral compound is introduced during step a), with the addition of the polysaccharide with the liquid enzymatic formulation and the support polymer into the mixer.

Many minerals may be used. Examples include calcite, carbonate salts or metal carbonates such as calcium carbonate, potassium carbonate, magnesium carbonate, aluminium carbonate, zinc carbonate, copper carbonate, chalk, dolomite; silicate salts, such as calcium silicate, potassium silicate, magnesium silicate, aluminium silicate, or a mixture thereof, like micas, smectites like montmorillonite, vermiculite, and sepiolite-palygorskite; sulphate salts, such as barium sulphate or calcium sulphate (gypsum), mica; hydroxide salts or metal hydroxides like calcium hydroxide, potassium hydroxide (potash), magnesium hydroxide, aluminium hydroxide, sodium hydroxide (caustic soda), hydrotalcite; metal oxides or oxides salts like magnesium oxide, calcium oxide, aluminium oxide, iron oxide, copper oxide, clay, asbestos, silica, graphite, carbon black; metal fibres or metal petals; glass fibres; magnetic fibres; ceramic fibres and derivatives and/or mixtures thereof.

In a preferred embodiment, the used mineral filler is calcium carbonate.

In general, the masterbatch is formulated with:
60 to 90% of support polymer, in particular PCL,
10 to 20% of liquid enzymatic formulation, in particular enzymatic solution,
2 to 15% of polysaccharide, in particular gum arabic, and
0 to 20% mineral filler, in particular calcium carbonate.

The masterbatch may also comprise the presence of one or more compound(s). In particular, the masterbatch may comprise one or more additive(s). In general, the additives are used in order to improve specific properties of the end product. For example, the additives may be semected from among plasticisers, colouring agents, processing aids, rheological agents, antistatic agents, anti-UV agents, reinforcement agents, compatibility agents, flame retardants, antioxidants, pro-oxidants, light stabilisers, oxygen scavengers, adhesives, products, excipients, etc.

Advantageously, the masterbatch comprises less than 20% by weight of additives and preferably less than 10% with respect to the total weight of the masterbatch. In general, the composition of the masterbatch comprises from 0% to 10% by weight of additives with respect to the total weight of the masterbatch.

The composition of the masterbatch after formulation comprises between 5% and 30% by weight of liquid enzymatic formualtion as defined hereinabove, with respect to the total weight of the masterbatch. In one embodiment, the enzymatic formulation represents between 8% and 22% by weight with respect to the total weight of the composition. In a preferred embodiment, the masterbatch comprises between 10% and 20% enzymatic formulation, by weight of its composition.

The method for manufacturing the masterbatch is carried out in a mixer. A person skilled in the art knows different types of mixers that could be used for the manufacture of these polymer masterbatches.

In a preferred embodiment, the mixer is an extruder. This may be of the single-screw or twin-screw type. Preferably, it is of the twin-screw type.

In particular, the method is implemented in an extruder comprising at least 3 areas, a head area where the first components are introduced, a mixing area and an outlet area through which the masterbatch is recovered, with the following steps a) to c):
a) separately and simultaneously introducing a polysaccharide and an enzymatic formulation in the head area, and where appropriate, a mineral filler, as defined hereinabove,
b) mixing the components in the mixing area at a temperature at which the support polymer is partially or totally molten,
c) recovering the masterbatch at the outlet of the extruder.

A person skilled in the art will know how to adapt the characteristics of the extruder (i.e. the length and diameter of the one or more screw(s), the degassing areas, etc.) and the stay time of the polysaccharide, the enzymes and the support polymer according to the time and temperature constraints of the different steps of the method of the invention.

In particular, the temperature at the head area is lower than the melting point of the support polymer and the temperature of the mixing area is higher than the temperature of the head area, in particular as defined hereinabove for mixing temperatures.

The mixing area itself could comprise several areas and a person skilled in the art could, where needed, adapt the temperatures of each area in particular according to the used enzymes and support polymers.

The masterbatch may be obtained in the form of granules prepared according to the usual techniques. These granules could be stored, transported and used in the manufacture of items made of a biodegradable plastic material, which may be called “end items”.

When it is in the form of granules, the masterbatch may be dried for storage thereof. The drying methods are the usual methods known to a person skilled in the art, in particular using hot air ovens, vacuum ovens, desiccators, microwaves or fluidised beds. The drying temperature and duration will depend on the one hand on the water content brought in by the enzyme solution in the preparation of the masterbatch, but also on the melting and glass transition temperatures of the used support polymer.

Once dry, the composition of the masterbatch advantageously comprises:
60% to 95% support polymer,
0.5% to 7% enzyme,
2% to 27% polysaccharide, and
0% to 30% mineral filler.

In general, the moisture content is 0.5% or less and preferably lower than 0.3%.

Afterwards, the obtained masterbatch in the form of granules may be used in the manufacture of biodegradable plastic products or “end items”. These may consist of films, or flexible or solid parts with shapes and volumes suited for their uses.

The biodegradable plastic item is obtained by mixing the masterbatch comprising the enzymes with at least one polymer able to be degraded by said enzymes.

Hence, the invention relates to a method for preparing an item made of a plastic material or a pre-mixture as defined hereinbelow comprising a polymer able to be degraded by enzymes and enzymes capable of degrading said polymer, said method comprising the steps of preparing a masterbatch comprising enzymes capable of degrading said polymer, a polysaccharide, and a support polymer, and possibly a mineral filler, the masterbatch being prepared in a mixer by a method comprising the following steps:
a) separately and simultaneously introducing, into the mixer, the enzymes in liquid formulation, the polysaccharide and the support polymer and, where appropriate, a mineral filler, as defined hereinabove
b) mixing the components, and
c) recovering the masterbatch,
   then mixing said polymer able to be degraded by enzymes with the masterbatch.

Advantageously, said polymer able to be degraded by the enzymes is a biodegradable polyester. These polyesters are well-known to a person skilled in the art, like polylactic acid (PLA), polyglycolic acid (PGA), polyhydroxyalkanoate (PHA), polycaprolactone (PCL), polybutylene succinate (PBS), polybutylene succinate adipate (PBSA), polybutylene adipate terephthalate (PBAT), plasticised starch and mixtures thereof.

These polyesters are selected for their physico-chemical properties according to the end item and the properties that are pursued, in particular its mechanical properties but also their colour and their transparency.

The biodegradable polyesters used for the preparation of end items have physicochemical properties identical to or different from the polyesters used as support polymers in the masterbatch according to the invention.

In a preferred embodiment, the polyester that could be degraded by the enzymes comprises PLA, alone or mixed with another polyester hereinabove, in particular a PLA/PBAT mixture.

Thus, the biodegradable plastic item consists of the masterbatch and a biodegradable polymer.

In addition to the biodegradable polymer, the composition of the biodegradable plastic item comprises 0.5% to 20% of enzyme masterbatch.

The methods for preparing these end items are well-known to a person skilled in the art, including in particular usual plastics techniques such as inflation extrusion, extrusion blow moulding, cast film extrusion, calendering and thermoforming, injection moulding, compression moulding, rotational moulding, coating, stratification, expansion, pultrusion and compression-granulation. Such operations are well-known to a person skilled in the art, who will easily adapt the conditions of the process according to the considered type of plastic items (for example the temperature, the stay time, etc.).

For the preparation of biodegradable plastic items, it is possible to mix the masterbatch with the other constituents of the composition for shaping thereof. It is also possible to prepare a pre-mixture or “compound” comprising the masterbatch and at least the biodegradable polymer. This pre-mixture in a solid form, in particular in the form of granules, may be stored and then transported before being used to form the end item, alone or associated with other constituents, according to the end composition of the end item.

Advantageously, the pre-mixture comprises:
8% to 99% by weight of biodegradable polymer, preferably PLA,
0.01% to 5% by weight of a polysaccharide, preferably a natural gum such as gum arabic,
0.1% to 20% by weight of a support polymer, as defined above, in particular PCL, and
0.01% to 2% by weight of enzymes having a biodegradable polymer degrading activity, more particularly having a PLA degrading activity, and where appropriate
0 to 35% by weight of mineral filler.

The end items may consist of films, flexible or solid parts with shapes and volumes suited to their uses. Examples of biodegradable plastic items concerned by the invention include films, mulching films, wrapping films, food or non-food films; packaging such as packaging blisters, trays; disposable tableware like cups, dishes or cutlery; stoppers and lids; beverage capsules; and horticultural items.

Advantageously, the composition of the plastic item is as follows:
60% to 98% by weight of polymer or mixture of biodegradable polymers,
0.01% to 5% by weight of a polysaccharide, preferably a natural gum such as gum arabic,
0.01% to 20% by weight of a support polymer, as defined hereinabove,
0.01% to 2% by weight of enzymes having a biodegradable polymer degrading activity,
0% to 35% by weight of mineral filler,
0% to 5% by weight of additives.

The biodegradable plastic items obtained with the enzyme masterbatch may be flexible and/or rigid.

In the case of flexible items, the polyester that can be degraded by enzymes comprises PLA. In one embodiment, the biodegradable polyester is a PBAT/PLA mixture the weight ratio of which preferably ranges from 10/90 to 20/80, more preferably from 13/87 to 15/85.

In another embodiment, the biodegradable polyester is a PBAT/PLA mixture the weight ratio of which ranges from 10/90 to 30/70, from 10/90 to 40/60, from 10/90 to 50/50, from 10/90 to 60/40, from 10/90 to 70/30, from 10/90 to 80/20, from 10/90 to 90/10.

In another embodiment, the biodegradable polyester is a PBAT/PLA mixture the weight ratio of which is less than 10/90, equal to or less than 9/91, equal to or less than 8/92, equal to or less than 7/93, equal to or less than 6/94, equal to or less than 5/95, equal to or less than 4/96, equal to or less than 3/97, equal to or less than 2/98, equal to or less than 1/99.

In another embodiment, the biodegradable polyester is PLA.

The flexible biodegradable plastic items are characterised by a thickness smaller than 250 μm, preferably by a thickness smaller than 200 μm. In a preferred embodiment, the films have a thickness smaller than 100 μm, more advantageously smaller than 50 μm, 40 μm or 30 μm, preferably between 10 and 20 μm. More preferably, the thickness of the flexible item is 15 μm. Examples include films, such as food films, wrapping films, industrial films or mulching films and bags.

Advantageously, the composition of the flexible item comprises:
70% to 98% by weight of polymer or a mixture of biodegradable polymer(s),
0.01% to 5% by weight of a polysaccharide, preferably a natural gum such as gum arabic,
0.1% to 20% by weight of a support polymer, as defined hereinabove, and
0.01% to 2% by weight of enzymes having a biodegradable polymer degrading activity,
0% to 5% by weight of mineral filler, in particular 0.01% to 5% by weight, in particular 0.05 to 5% by weight,
0% to 5% by weight of additives.

The composition according to the invention is particularly suitable for making plastic films. The films according to the invention may be produced according to the usual technical methods, in particular by inflation extrusion. The films may be prepared from granules having a composition according to the invention, which are melted according to the usual techniques, in particular by extrusion.

The films having a composition as previously defined with enzymes may consist of single-layer or multilayer films. In the case of a multilayer film, at least one of the layers has a composition as previously defined. The single-layer and multilayer films, having a composition as previously defined, have a high PLA content and keep mechanical properties as desired for the preparation of biodegradable and bio-sourced films, in particular for packaging food and non-food products. To this end, the constituents of the composition according to the invention will be preferably selected from among products compatible with food use.

The multilayer film may be a film comprising at least 3 layers, of the ABA, ABCA or ACBCA type, the layers A, B and C having different compositions. In a preferred embodiment, the multilayer films are of the ABA or ACBCA type.

In general, the layers A and B comprise PLA and/or a polyester, advantageously a composition according to the invention. The layers C, if present, are there in order to confer particular properties on the items according to the invention, more particularly to conf gas, in particular oxygen, barrier properties. Such barrier materials are well-known to a person skilled in the art, and in particular PVOH (polyvinyl alcohol), PVCD (polyvinyl chloride), PGA (polyglycolic acid), cellulose and derivatives thereof, milk proteins or polysaccharides and mixtures thereof in all proportions.

In the case of multilayer films as defined hereinabove, and in particular for films of the ABA, ABCA or ACBCA type, the enzymes may be present in all layers or in only one of the layers, for example in the layers A and B or only in the layer A or in the layer B.

According to a particular embodiment of the invention, the two layers A consist of a composition according to the invention comprising PLA, polyester and polypropylene glycol diglycidyl ether (PPGDGE), without enzymes. The enzymes are in the layer B, either in a composition according to the invention with enzymes as defined hereinabove, or in a particular composition, in particular an enzyme composition in a low-melting polymer defined hereinabove.

According to the embodiments, the composition of the enzymatic layer of the (single-layer or multilayer) flexible items may comprise up to 95% by weight of biodegradable polymer, preferably PLA. Hence, the enzymatic layer may comprise 8% to 50%, 8% to 60%, 8% to 70%, 8% to 80% or 8% to 90% by weight of biodegradable polymer.

Advantageously, the composition of the enzymatic layer of (single-layer or multilayer) flexible items comprises:
8% to 95% by weight of biodegradable polymer, preferably PLA, in particular 8% to 70%, 8% to 60%, 8% to 50%, or 8% to 40%,
0.02% to 4% by weight of a polysaccharide, preferably a natural gum such as gum arabic
0.1% to 19% by weight of a support polymer, as defined hereinabove, and
0.05% to 2% by weight enzymes having a biodegradable polymer degrading activity, more particularly having a PLA degrading activity, and where appropriate
0% to 5% by weight of mineral filler, in particular 0.01% to 5% by weight, in particular 0.05 to 5% by weight.

As regards rigid items, the biodegradable polyester is PLA, preferably a PLA/calcium carbonate mixture the weight ratio of which ranges from 100/0 to 25/75, preferably from 95/5 to 45/55, more preferably from 90/10 to 50/50. In another embodiment; the biodegradable polyester is a PBAT/PLA mixture the weight ratio of which preferably ranges from 10/90 to 80/20, more preferably from 20/80 to 60/40.

The rigid items have a thickness comprised between 200 μm and 5 mm, between 150 μm and 5 mm, preferably between 200 μm and 3 mm, or between 150 μm and 3 mm. In one embodiment, the items have a thickness comprised between 200 μm and 1 mm, between 150 μm and 1 mm, preferably between 200 μm and 750 μm or between 150 μm and 750 μm. In another embodiment, the thickness is 450 μm.

Examples of such biodegradable plastic items include cups, dishes, cutlery, trays, drink capsules and packaging blisters, more generally packaging for food, cosmetics or horticultural products.

Advantageously, the composition of the rigid item comprises:
60% to 95% by weight of a polymer or a mixture of biodegradable polymer(s),
0.01% to 5% by weight of a polysaccharide, preferably a natural gum such as gum arabic,
0.1% to 20% by weight of a support polymer, as defined hereinabove,
0.01% to 2% by weight of enzymes having a biodegradable polymer degrading activity,
0% to 35% by weight of mineral filler, in particular 0.01 to 35% by weight,
0% to 5% by weight of additives.

In another embodiment, the composition of the rigid item comprises:
60% to 80% by weight of a polymer or a mixture of biodegradable polymer(s),
0.01% to 5% by weight of a polysaccharide, preferably a natural gum such as gum arabic,
0.1% to 20% by weight of a support polymer, as defined hereinabove, and
0.01% to 2% by weight of enzymes having a biodegradable polymer degrading activity,
8% to 35% by weight of mineral filler,
0% to 5% by weight of additives.

Thus, the composition of the rigid item comprises more than 60% by weight of biodegradable polymer or mixture of polymer(s), or more than 70%, or more than 80%, or more than 90%.

The mineral filler content in the rigid item is comprised between 0.01% and 35% by weight according to the nature of the mineral filler.

According to some embodiments, the rigid item thus comprises more than 0.01%, more than 0.1%, more than 1%, or more than 2%, or more than 3% by weight of mineral filler.

In other embodiments, the amount by weight of mineral filler is greater than or equal to 4%, greater than or equal to 5%, greater than or equal to 6%, greater than or equal to 7%, or greater than or equal to 8%.

In still other embodiments, the mineral filler comprised in the rigid item is 10 to 35% by weight, 15% to 30%, or 20% to 28% by weight.

Whether flexible or rigid, the end items may also comprise plasticisers, compatibilisers and other common additives included in the composition of plastic materials, like pigments or dyes, release agents, impact modifiers, anti-blocking agents, etc.

Examples of plasticisers include citrate esters and lactic acid oligomers (LAO).

Citrate esters are plasticisers known to a person skilled in the art, in particular as bio-sourced materials. Mention may in particular be made of triethyl citrate (TEC), the triethyl acetyl citrate (TEAC), tributyl citrate (TBC) and tributyl acetyl citrate (TBAC). Preferably, the citrate ester used as plasticiser in the composition according to the invention is TBAC.

LAOs are also plasticisers known to a person skilled in the art, in particular as bio-sourced materials. These consist of lactic acid oligomers having a molecular weight of less than 1,500 g/mol. Preferably, they are esters of lactic acid oligomers, their carboxylic acid termination being blocked by esterification with an alcohol, in particular a C1-C10 linear or branched alcohol, advantageously a C6-C10 alcohol, or a mixture thereof. Mention may in particular be made of the LAOs described in the patent application EP 2 256 149 with their preparation method, and of the LAOs commercialised by Condensia Quimica under the brand Glyplast®, in particular the references Glyplast® OLA 2, which has a molecular weight of 500 to 600 g/mol and Glyplast® OLA 8 which has a molecular weight of 1,000 to 1,100 g/mol. According to a preferred embodiment of the invention, the LAOs have a molecular weight of at least 900 g/mol, preferably 1,000 to 1,400 g/mol, more preferably 1,000 to 1,100 g/mol.

Poly(propylene glycol) diglycidyl ethers, also called glycidyl ethers, referred to in particular as “reactive plasticisers” in the patent application WO 2013/104743, used for the preparation of block copolymers with PLA and PBAT. They are also identified as liquid epoxy resin, from the company DOW, commercialised under the reference “D.E.R.™ 732P”, or as aliphatic epoxy resin, from the company HEXION, commercialised under the reference “Epikote™ Resin 877”.

Optionally, the composition according to the invention may comprise other PLA/polyesters compatibilisers associated with PPGDGE. Such PLA/polyesters compatibilisers are well-known to a person skilled in the art, in particular selected from among polyacrylates, ethylene terpolymers, acrylic ester and glycidyl methacrylate (for example commercialised under the brand Lotader® by the company Arkema), triblock copolymers PLA-PBAT-PLA, PLA grafted with maleic anhydride (PLA-g-AM) or PBAT grafted with maleic anhydride (PBAT-g-AM), in particular poly (ethylene-co-methyl acrylate-co-glycidyl methacrylate) described in particular by Dong & al. (International Journal of Molecular Sciences, 2013, 14, 20189-20203) and Ojijo & al. (Polymer 2015, 80, 1-17), more particularly commercialised under the name JONCRYL® by the company BASF, preferably the ADR 4468 grade.

### EXAMPLES

### Example 1: Use of the Masterbatch in Flexible Items

### I. Preparation of a Support Polymer and Enzymes Mixture

### 1. Preparation of a Masterbatch Under the Conditions of the Prior Art

The support polymer and enzymes mixture A1 is prepared from granules of polycaprolactone (PCL), a polysaccharide (gum arabic), a mineral filler (calcium carbonate, CaCO₃) and enzymes in solution. The support polymer and enzymes mixture has been manufactured with a co-rotating twin-screw extruder Clextral Evolum 25 HT comprising 11 areas for which the temperature is independently monitored and regulated. The enzymes in solution, the gum arabic and the calcium carbonate have been introduced simultaneously at the inlet of the extruder in order to carry out mixing according to an increasing temperature profile comprised between 25 and 50° C. The enzymes in solution are introduced at 2.6 kg/h using a peristaltic pump. In turn, the gum arabic is introduced at 1.4 kg/h using a batcher specific for powders and the calcium carbonate is introduced at 2 kg/h. The PCL, also called support polymer, is introduced at 14 kg/h in a partially or totally molten state between the area 5 and the area 6 of the extruder at a temperature of 75° C.

The mixture A2 is made under the same conditions as the mixture A1, on a different day.

The support polymer and enzymes mixture A3 is prepared from granules of polycaprolactone (PCL), a polysaccharide (gum arabic), a mineral filler (calcium carbonate, CaCO₃) and enzymes in solution. The support polymer and enzymes mixture has been manufactured with a co-rotating twin-screw extruder Clextral Evolum 25 HT comprising 11 areas for which the temperature is independently monitored and regulated. The enzymes in solution, the gum arabic and the calcium carbonate have been introduced simultaneously at the inlet of the extruder in order to carry out mixing according to an increasing temperature profile comprised between 25 and 50° C. The enzymes in solution are introduced at 2.2 kg/h using a peristaltic pump. In turn, the gum arabic is introduced at 1.8 kg/h using a batcher specific for powders and the calcium carbonate is introduced at 2 kg/h. The PCL, also called support polymer, is introduced at 14 kg/h in a partially or totally molten state between the area 5 and the area 6 of the extruder at a temperature of 75° C. The granulation of each mixture is done with cutting under water. The granules are dried at 45° C. with a moisture up to 0.3%.

### 2. Preparation of a Masterbatch According to the Method of the Invention

The support polymer and enzymes mixture B1 is prepared from granules of polycaprolactone (PCL), a polysaccharide (gum arabic) and enzymes in solution according to the method of the invention.

The support polymer and enzymes mixture has been manufactured with a twin-screw extruder CLEXTRALEvolum 25 HT comprising 11 areas for which the temperature is independently monitored and regulated according to an increasing temperature profile comprised between 25° C. and 50° C. The PCL, the enzymes in solution and the gum arabic are introduced separately and simultaneously at the head of the twin-screw. The PCL is introduced at 16 kg/h, the enzymes in solution are introduced at 2.2 kg/h using a peristaltic pump, the gum arabic is introduced at 1.8 kg/h using a batcher specific for powders.

The mixture B2 is made under the same conditions as the mixture B1, on a different day.

The support polymer and enzymes mixture B3 is prepared from granules of polycaprolactone (PCL), a polysaccharide (gum arabic), a mineral filler (calcium carbonate, CaCO₃) and enzymes in solution according to the method of the invention.

The support polymer and enzymes mixture has been manufactured with a twin-screw extruder CLEXTRALEvolum 25 HT comprising 11 areas for which the temperature is independently monitored and regulated according to an increasing temperature profile comprised between 25° C. and 60° C. The PCL, the enzymes in solution, the gum arabic and the calcium carbonate are introduced separately and simultaneously at the head of the twin-screw. The PCL is introduced at 14 kg/h, the enzymes in solution are introduced at 2.2 kg/h using a peristaltic pump, the gum arabic is introduced at 1.8 kg/h using a batcher specific for powders and the calcium carbonate is introduced at 2 kg/h.

### II. Commercial Products

In these examples, PCL marketed under the reference Capa™ 6500 by the company Perstorp, calcium carbonate commercialised under the reference OMYAFILM 707-OG by the company Omya, gum arabic commercialised under the reference InstantGum AA by the company Nexira have been used.

A PLA/PBAT mix commercialised under the reference ECOVIO F2223 by the company BASF has been used.

### III. Production of the Films

For inflation extrusion, a laboratory bench Labtech LF-250, laize 20 mm, with an LBE20-30/C type 30 L/D screw has been used. The screw speed is between 50 and 60 rpm, the top and low drawing speeds are between 4.3 and 5.7 m/min.

The inflation extrusion temperatures are detailed in the tables 1a, 1b and 1c.

TABLE 1a
Inflation extrusion temperatures for the films 1
and 2, respectively with the mixtures A1 and A2.

| Area | Z1 | Z2 | Z3 | Z4 | Extruder #1 | Extruder #2 |
|---|---|---|---|---|---|---|
| Temperature | 150 | 150 | 150 | 150 | 155 | 150 |
| (° C.) | | | | | | |

TABLE 1b
Inflation extrusion temperatures for the films 4
and 5, respectively with the mixtures B1 and B2.

| Area | Z1 | Z2 | Z3 | Z4 | Extruder #1 | Extruder #2 |
|---|---|---|---|---|---|---|
| Temperature | 125 | 165 | 155 | 155 | 160 | 160 |
| (° C.) | | | | | | |

TABLE 1c
Inflation extrusion temperatures for the films 3
and 6, respectively with the mixtures A3 and B3.

| Area | Z1 | Z2 | Z3 | Z4 | Extruder #1 | Extruder #2 |
|---|---|---|---|---|---|---|
| Temperature | 135 | 145- | 155 | 155 | 160 | 160 |
| (° C.) | | 150 | | | | |

The films have an average thickness of 15 μm. The thicknesses have been measured with a Positector electronic micrometer.

These films are transparent, with no roughness and no passing defect has been identified. The bubble was stable for all inflation extrusions. Opening of the film after inflation extrusion has been described as standard, with no difficulty.

### IV. Analysis Methods

The tensile and tear mechanical properties may be measured using a Zwick or Llyod type machine, equipped with a 50 N sensor or with a 5 kN sensor. The properties are measured in two different directions: in the longitudinal direction and in the transverse direction. The tensile and tear mechanical properties are measured according to the standards EN ISO 527-3 and ISO 6383-1 respectively.

As regards resistance to perforation, this is measured using a Dart-Test according to the standard NF EN ISO 7765-1.

The assessment of the biodegradability of the films has been assessed using a depolymerisation test performed according to the following protocol: 100 mg of each sample was introduced into a plastic vial containing 50 mL of a buffer solution at a pH 8. The depolymerisation is launched by incubating each sample at 45° C., in an incubator stirred at 150 RPM. A 1 mL aliquot of a buffer solution is regularly sampled and filtered using a 0.2 μm filter syringe in order to be analysed by high-performance liquid chromatography (HPLC) using an Aminex HPX-87H column to measure the release of lactic acid (LA) and its dimer. The used chromatography system is a HPLC Nexan Series, SHIMADZU machine comprising a pump, an automatic sampler, a column thermostated at 50° C. and a 220 nm UV detector. The eluent is a H₂SO₄ solution at 5 mM. The injection amounts to 20 μL of sample. The lactic acid is measured based on standard curves prepared from commercial lactic acid.

The hydrolysis of the plastic films is calculated based on the lactic acid and the lactic acid dimer released. The depolymerisation percentage is calculated with regards to the PLA percentage in the sample.

### V. Analysis Results

### Granule Density by Pycnometry

The masterbatch A1 obtained with the preparation method of the prior art described in paragraph I.1 has a density equivalent to that of the masterbatch B1 obtained with the preparation method of the invention described in paragraph I.2, namely 1.03 g/cm³ in average.

The masterbatch A2 obtained with the preparation method of the prior art described in paragraph I.1 has a density equivalent to that of the masterbatch B2 obtained with the preparation method of the invention described in paragraph I.2, namely 1.05 g/cm³ in average.

The masterbatch A3 obtained with the preparation method of the prior art described in paragraph I.1 has a density equivalent to that of the masterbatch B3 obtained with the preparation method of the invention described in paragraph I.2, namely 1.1 g/cm³ in average.

The preparation method of the support polymer and enzymes masterbatch has no impact on the density of the end compound.

### Melt Flow Index (MFI) Measurements

The masterbatch A1 has a melt flow index equivalent to that of the masterbatch B1, namely 10-10.5 for an analysis carried out at 160° C. and 2.16 kg.

The masterbatch A3 has a melt flow index equivalent to that of the masterbatch B3, namely 10-10.5 for an analysis carried out at 160° C. and 2.16 kg.

The preparation method of the support polymer and enzymes masterbatch has no impact on the fluidity of the end compound.

### Thermogravimetric Analyses

The thermogravimetric analyses carried out on these two mixtures prepared in paragraph I show that all of the components of the formulation are at equivalent decomposition temperatures.

TABLE 2
Results of the thermogravimetric analysis

| Decomposition | | |
|---|---|---|
| temperature | Mixture A1 | Mixture B1 |
| ~305° C. | ~6.8% | ~7.6% |
| ~390° C. | ~74.2% | ~86% |
| ~450° C. | ~8.5% | ~4% |
| Residues | ~10.6% | ~2.9% |
| Decomposition | | |
| temperature | Mixture A2 | Mixture B2 |
| ~300° C. | ~7.4% | ~7.5% |
| ~390° C. | ~72.4% | ~90% |
| ~420° C. | ~9.1% | ~0% |
| Residues | ~11.1% | ~2.4% |
| Decomposition | | |
| temperature | Mixture A3 1 | Mixture B3 |
| ~300° C. | ~7.9% | ~8.2% |
| ~390° C. | ~70.6% | ~70.4% |
| ~450° C. | ~7.5% | ~7% |
| Residues | ~14.1% | ~14% |

The formulations of the masterbatches described in paragraph I.1 (prior art) and I.2 (invention) feature composition differences between the compounds A1 and B1; A2 and B2 as well as A3 and B3. These differences are observed in terms of mass losses upon the thermogravimetric analysis.

Only the masterbatches A3 and B3 have the same compositions and the mass losses do not feature significant differences according to the used method.

### Composition of the Films

The films have been prepared with the support polymer and enzymes mixtures A1-A2-A3-B1-B2-B3 prepared in I.1 and I.2 and a PLA-and PBAT-based grade commercialised under the reference ECOVIO F2223 by the company BASF and referred to as “Compound 1” in the examples hereinbelow.

The compositions of these different films are reported in Table 3.

TABLE 3
Summary of the single-layer films made

| | Film composition | Enzyme total content (%) |
|---|---|---|
| Film 1 | Compound 1 + Mixture A1 | >0.15% and identical to the film 2 |
| Film 2 | Compound 1 + Mixture A2 | >0.15% and identical to the film 1 |
| Film 3 | Compound 1 + Mixture A3 | >0.1% |
| Film 4 | Compound 1 + Mixture B1 | >than the % of the enzyme of the |
| | | films 1 et 2 and equivalent to the |
| | | film 5 |
| Film 5 | Compound 1 + Mixture B2 | >than the % of the enzyme of the |
| | | films 1 et 2 and equivalent to the |
| | | film 4 |
| Film 6 | Compound 1 + Mixture B3 | >0.1% and identical to the film 3 |

The film 1 serves as a reference of the prior art for the film 4 of the invention.

The film 2 serves as a reference of the prior art for the film 5 of the invention.

The film 3 serves as a reference of the prior art for the film 6 of the invention.

The manufacturing method of the masterbatch has no impact on the inflation extrusion process. The parameters of the inflation extrusion remain identical between the film of the prior art and of the invention. Regardless of the used manufacturing process, the appearance of the films is identical.

### Mechanical Properties of the Films 3 (Prior Art) and 6 (Invention)

The mechanical properties of the films 3 and 6 obtained with masterbatches having identical compositions have been measured. The results are reported in Table 4. The indicated values represent the average of all of the completed measurements.

TABLE 4
Characterisation of the mechanical properties of the films

| Film | | 3 | 6 |
|---|---|---|---|
| film density (g/cm³) film | | 1.23 | 1.23 |
| thickness (μm) | | 12 | 11 |
| Properties | Measurement | | |
| | direction | | |
| Tensile strength (%) | LD | 100% | +0% |
| | TD | 100% | −7% |
| Elongation at break (%) | LD | 100% | −21% |
| | TD | 100% | +47% |
| Young's modulus (%) | LD | 100% | 24% |
| | TD | 100% | +11% |
| Tear resistance (%) | TD | 100% | +0% |

(with LD = Longitudinal direction of the film and TD = transverse direction of the film)

The mechanical properties thus measured show that the film 6 according to the invention has mechanical properties equivalent to or greater than those of the film 3 according to the prior art.

### Depolymerisation of the PLA of the Films

The films 1 and 2 containing a support polymer and enzymes mixture manufactured under conventional conditions, and with the same enzymes content have depolymerisation results comprised between 34 and 38% after 20 days at 28° C. The films 4 and 5, containing a support polymer and enzymes mixture manufactured according to the method described in the invention and with the same enzymes content but lower than the films 1 and 2 have depolymerisation results between 28% and 31%.

The films 3 and 6 with an identical enzymes content and with MBs manufactured by the two processes have a depolymerisation rate comprised between 65% and 77% after 5 days at 45° C. The addition of calcium carbonate in the composition of the support polymer and enzyme mixture promotes the depolymerisation of PLA. In turn, the preparation method of the masterbatch has no negative impact on the performance of the enzymes in the masterbatch. The preparation method of the mixture described in the invention allows reaching depolymerisation rates close to the conventional method but with less enzymes.

### Example 2: Use of the Masterbatch in Flexible Items

### I. Preparation of a Support Polymer and Enzymes Mixture

### 1. Preparation of a Masterbatch Under Conditions of the Prior Art (With a Formulated Enzymatic Solution)

The support polymer and enzymes mixture A4 is prepared from granules of polycaprolactone (PCL) and enzymes in solution formulated with a polysaccharide (gum arabic). The support polymer and enzymes mixture A5 is prepared from granules of polycaprolactone (PCL), a mineral filler (calcium carbonate, CaCO₃) and enzymes in solution formulated with a polysaccharide (gum arabic). The masterbatches A4 and A5 are prepared according to the procedure of Example 1.I.1 with an increasing temperature profile comprised between 30 and 65° C.

For the mixture A4, the PCL is introduced at the head of the twin-screw at 17 kg/h, the enzymes in the solution formulated with arabica gum are introduced at 3 kg/h using a peristaltic pump.

For the mixture A5, the PCL is introduced at the head of the twin-screw at 17 kg/h, the enzymes in the solution formulated with arabica gum are introduced at 3 kg/h using a peristaltic pump. Calcium carbonate is introduced at 2 kg/h using a batcher specific for powders.

### 2. Preparation of a Masterbatch According to the Method of the Invention (With a Non-Formulated Enzymatic Solution)

The support polymer and enzymes mixture B4 is prepared from granules of polycaprolactone (PCL), a polysaccharide (arabica gum) and enzymes in solution.

The support polymer and enzymes mixture B5 is prepared from granules of polycaprolactone (PCL), a polysaccharide (arabica gum), a mineral filler (calcium carbonate, CaCO₃) and enzymes in solution.

The masterbatches B4 and B5 are prepared according to the procedure of Example 1.I.2 with an increasing temperature profile comprised between 30 and 65° C.

For the mixture B4, the enzymes in solution are introduced at 2.2 kg/h using a peristaltic pump, the arabica gum is introduced at 0.8 kg/h using a batcher specific for powders and PCL is introduced at 17 kg/h at the head of the twin-screw.

For the mixture B5, the enzymes in solution are introduced at 2.2 kg/h using a peristaltic pump, the gum arabic is introduced at 0.8 kg/h using a batcher specific for powders, the calcium carbonate is introduced at 2 kg/h and PCL is introduced at 15 kg/h at the head of the twin-screw. The granulation of each mixture is done with cutting under water. The granules are dried at 45° C. with a moisutre up to 0.3%.

### II. Commercial Products

The products used for the preparation of the masterbatches and of the films are those used for Example I.

### III. Production of the Films

The films 7 and 8 prepared respectively with the masterbatches A4 (prior art) and B4 (obtained according to the invention) are prepared according to the methods described for the films of Example 1.III.

### IV. Analysis Methods

The analysis methods are those described for Example 1.IV.

### V. Analysis Results

### Granule Density by Pycnometry

The masterbatch A4 (prior art) has a density equivalent to that of the masterbatch B4 obtained according to the invention, namely 1.06 g/cm³ in average.

The masterbatch A5 (prior art) has a density equivalent to that of the masterbatch B5 obtained according to the invention, namely 1.4 g/cm³ in average.

The preparation method of the support polymer and enzymes masterbatch has no impact on the density of the end compound.

### Melt Flow Index (MFI) Measurements

The masterbatch A4 has a melt flow index equivalent to that of the masterbatch B4 obtained with the preparation method of the invention, namely 15.6 and 14.1 g/10 min respectively for an analysis carried out at 160° C. and 2.16 kg.

The masterbatch A5 has a melt flow index equivalent to that of the masterbatch B5 obtained with the preparation method of the invention, namely 18.3 and 18.9 g/10 min respectively for an analysis carried out at 160° C. and 2.16 kg.

The preparation method of the support polymer and enzymes masterbatch has no impact on the fluidity of the end compound.

### Thermogravimetric Analyses

The thermogravimetric analyses carried out on the masterbatches prepared in paragraph 2.I show that all of the components of the formulation are at equivalent decomposition temperatures.

TABLE 5
Results of the thermogravimetric analysis

| Decomposition | | |
|---|---|---|
| temperature | Mixture A4 | Mixture B4 |
| ~305° C. | ~4.2% | ~4.5% |
| ~400° C. | ~91.8% | ~90.5% |
| Residues | ~0.53% | ~0.16% |
| Decomposition | | |
| temperature | Mixture A5 | Mixture B5 |
| ~300° C. | ~3.8% | ~5.3% |
| ~380° C. | ~78.9% | ~78.6% |
| ~455° C. | ~3.6% | ~3.6% |
| ~665° C. | ~5.4% | ~4.9% |
| Residues | ~6.8% | ~6.5% |

The masterbatches A4 and A5 (prior art) have equivalent mass losses at temperatures similar to those of the masterbatches B4 and B5 obtained according to the invention.

The use of an enzymatic solution formulated according to the prior art or non-formulated according to the invention during the preparation of the masterbatches has no impact on the mass losses during the thermogravimetric analysis.

### Composition of the Films

The films have been prepared with the polymer support and enzymes masterbatches A4 and B4 and a PLA and PBAT based grade commercialised under the reference ECOVIO F2223 by the company BASF and referred to as “Compound 1” in the examples hereinbelow.

The compositions of these different films are reported in Table 9.

TABLE 6
Summary of the single-layer films made

| | Film composition | Enzyme total content (%) |
|---|---|---|
| Film 7 | Compound 1 + Mixture A4 | ~0.097% |
| Film 8 | Compound 1 + Mixture B4 | |

The film 7 serves as a reference of the prior art for the film 8 of the invention.

The manufacturing method of the mixture has no impact on the inflation extrusion process. The parameters of the inflation extrusion process remain identical between the film of the prior art and of the invention. Regardless of the used manufacturing process, the appearance of the films is identical.

### Mechanical Properties of the Films 7 and 8

The measured mechanical properties are reported in Table 7. The indicated values represent the average of all of the completed measurements.

TABLE 7
Characterisation of the mechanical properties of the films 7 and 8

| Film | | 7 | 8 |
|---|---|---|---|
| film density (g/cm³) film | | 1.25 | 1.25 |
| thickness (μm) | | 17.3 | 16.8 |
| | Measurement | | |
| Properties | direction | | |
| Tensile strength (%) | LD | 100% | +27% |
| | TD | 100% | 24% |
| Elongation at break (%) | LD | 100% | +14% |
| | TD | 100% | 24% |
| Young's modulus (%) | LD | 100% | +11% |
| | TD | 100% | +15% |
| Tear resistance (%) | LD | 100% | +4.2% |
| | TD | 100% | +5.2% |
| Resistance to perforation - | | 100% | +80% |
| Dart test (%) | | | |

(with LD = Longitudinal direction of the film and TD = transverse direction of the film)

The measured mechanical properties show that the film 8 obtained with the masterbatch B4 according to the invention has mechanical properties that are superior than the film 7 obtained with the masterbatch A4 according to the prior art.

1. A method for preparing a masterbatch comprising a polysaccharide, enzymes capable of degrading polyesters and a support polymer in a mixer,
characterised in that said method comprises the following steps:
a) separately and simultaneously introducing a liquid enzymatic formulation, polysaccharide and the support polymer,
b) mixing them at a temperature at which the support polymer is partially or totally molten, and
c) recovering the masterbatch after mixing.

2. The method according to claim 1, characterised in that the polysaccharide is selected from among starch derivatives, natural gums, soluble soybean polysaccharides, marine extracts and microbial and animal polysaccharides, or mixtures thereof in any proportions.

3. The method according to claim 1, characterised in that the polysaccharide consists of gum arabic.

4. The method according to claim 1, characterised in that the enzymes are added in the form of an aqueous solution.

5. The method according to claim 1, characterised in that the enzymatic formulation comprises 0.01 to 70% by weight of enzymes.

6. The method according to claim 1, characterised in that the enzymes are selected from among enzymes capable of degrading the polyesters selected from among depolymerases, esterases, lipases, cutinases, carboxylesterases, proteases and polyesterases.

7. The method according to claim 1, characterised in that the support polymer is selected from among polycaprolactone (PCL), polybutylene succinate (PBS), polybutylene succinate adipate (PBSA), polybutylene adipate terephthalate (PBAT), polydioxanone (PDS), polyhydroxyalkanoate (PHA) and polylactic acid (PLA) and mixtures thereof.

8. The method according to claim 1, characterised in that mixing in step b) lasts between 10 and 35 seconds.

9. The method according to claim 1, characterised in that it comprises simultaneously adding a mineral filler in step a), in particular calcium carbonate.

10. The method according to claim 1, characterised in that the mixer is an extruder.

11. The method according to claim 10, characterised in that the extruder comprises at least 3 areas, a head area where the first components are introduced, a mixing area and an outlet area through which the masterbatch is recovered, with the following steps a) to c):
a) separately and simultaneously introducing a liquid enzymatic formulation, a polysaccharide and a support polymer and possibly a mineral filler in the head area, and mixing them at a temperature lower than or equal to the melting point of the support polymer,
b) mixing the components in the mixing area at a temperature at which the support polymer is partially or totally molten, and
c) recovering the masterbatch at the outlet of the extruder.

12. The method according to claim 1, characterised in that the masterbatch is obtained in step c) in the form of granules.

13. The method according to claim 12, characterised in that the masterbatch mixture are dried.

14. The method according to claim 1, characterised in that the formulation of the masterbatch contains
60 to 90% of a support polymer,
10 to 20% of an enzymatic solution,
2 to 15% of polysaccharide,
0 to 20% of mineral filler.

15. A method for preparing a plastic item or a pre-mixture comprising a polymer that is able to be degraded by enzymes and enzymes capable of degrading said polymer, characterised in that it comprises a step of preparing a masterbatch according to claim 1, and a step of mixing the masterbatch previously prepared with the polymer, the enzymes of the masterbatch being capable of degrading said polymer of the plastic item or of the pre-mixture.

16. The method according to claim 15, characterised in that the polymer that could be degraded by enzymes of the plastic item or of the pre-mixture is polylactic acid (PLA).

17. The method according to claim 7, characterised in that the support polymer is polycaprolactone (PCL).

18. The method according to claim 8, characterised in that mixing in step b) lasts between 15 and 35 seconds.

19. The method according to claim 18, characterised in that mixing in step b) lasts for about 20 seconds, for about 25 seconds or for about 30 seconds.
